(19) United States
(12) Patent Application Publication
(10) Pub. No.: US 2026/0041760 A1
(43) Pub. Date: Feb. 12, 2026

(54) IMMUNE PROPHYLAXIS, THERAPY AND VACCINE FOR COVID-19 AND ITS EMERGING VARIANTS

(71) Applicant: Kumarpal A. SHAH, Searingtown, NY (US)

(72) Inventor: Kumarpal A. SHAH, Searingtown, NY (US)

(21) Appl. No.: 18/800,016

(22) Filed: Aug. 10, 2024

(51) Int. Cl. A61K 39/215; G06F 30/27; G16H 10/20; G16H 20/10

(52) U.S. Cl. CPC A61K 39/215; G06F 30/27; G16H 10/20; G16H 20/10

(57) ABSTRACT

Prophylaxis, immune therapy and vaccine strategies for Covid-19 variants and comorbid conditions such as aging, diabetes, cancer, tuberculosis or HIV. In one mode of invention, the role of the C3 Amplification loop is defined and how it derails the SNS functions or immune function at fluid and tissue levels. The plasticity in subverting C3 amplification control mechanism contribute to survival strategies of SARS-CoV-2 that contributes to various variants that can be reactivated at any stage of life based on immune system of patient that may get compromised with comorbid diseases such as aging, diabetes, cancer and HIV. Therapeutic modulation takes into consideration plasticity of different patient needs and their comorbid conditions by developing varying formulation methods and its combination approaches.

### BACKGROUND OF THE INVENTION

### 1. Field of the Invention

The invention relates to immune prophylaxis, therapy and vaccine for Covid-19 and its emerging variants.

### 2. the Prior Art

The Control of Covid-19 pandemics with its new variants is assuming new global urgency from public health point of view. This goal can only be achieved with novel approaches in drugs and vaccines development that has potential to boost host immunity. Such understanding is deeply embedded in “The Self-Non Self” (SNS) concept of earliest human defense system that educates, primes and modulate adaptive immune responses. This invention applies artificial intelligence “Tool-Kit” to published literature database and convert it into novel actionable insight that defines a. new immune pathogenesis, b. new molecular targets; c. Novel SNS mimicking compound that:

In one mode of invention “Artificial Intelligence Tool Kit of SNS modeling” identifies Immune evasion of C3 Amplification loop by SARS-CoV-2 as basic immune pathogenesis of Covid-19 and its variants that contribute host inflammatory destruction of multiple organs.

In another mode of invention “Artificial Intelligence Tool Kit of SNS modeling applied to identify key proteins that regulate “C3 amplification loop” and its downward and upward regulations of immune dysfunctions.

In yet another mode of invention “Artificial Intelligence Tool Kit of SNS modeling” applied for throughput chemical screening to identify “SNS mimicking decoy compound that control both downward and upward regulators of “C3Amplification Loop”.

Most specifically, the invention details sulfonic polymers and its formulations to intercept immunosuppressive, immune evasive and immunoinflammatory conditions caused by SARS-CoV-2 to cause Covid-19 and its emerging deadly variants by targeting upward and downward regulators of “C3 Amplification loop” as a method to provide immune prophylaxis, therapy and Vaccine responses.

Xiao, M. T. et al, Emerging role of complement in Covid-19 and other respiratory diseases, published their review and findings in “Cellular and Molecular Life sciences, 81:94, 2024 (doi.org/10.1007/s00018-024-05157-8) (1). In the abstract they state “The Complement system, a key component of innate immunity, provides the first line defense against bacterial infection; however, the Covid-19 pandemic has revealed that it may also engender severe complications in the context viral respiratory disease. In above article they reviewed the mechanism of complement activation and regulation and explore their roles in both protecting against infection and exacerbating disease. They discuss emerging evidence related to complement-targeted therapeutics in COVID-19 and compare the role of the complement in other respiratory viral diseases like influenza and respiratory syncytial virus. They also reviewed recent mechanistic studies and animal models that can be used for further investigation. Novel knockout studies are proposed to better understand the nuances of the activation of the complement system in respiratory viral diseases.”

Covid-19 pandemic and its variants is a cause of great concern to entire population of the world as virulent form will strike large scale mortality of patients, healthcare workers and will grind holt to global economic activities and democracies.

Despite effective control measures such as public education, quarantine of COVID-19 patients, early therapy with AntiCovid-19 drugs such as Paxlovid and various vaccines Covid-19 continues to be number one infectious pandemic disease globally. Elflein J in May 2024, published data of Covid-19 deaths worldwide as of May 2, 2023 by country and territory COVID-19 deaths worldwide as of May 2, 2023, by country and territory (statista.com/statistics/1093256/novel-coronavirus-2019ncov-deaths-worldwide-by-country/). (2)

As of May 2, 2023, the outbreak of the coronavirus disease (COVID-19) had spread to almost every country in the world, and more than 6.86 million people had died after contracting the respiratory virus. Over 1.16 million of these deaths occurred in the United State. Almost every country and territory worldwide have been affected by the COVID-19 disease (statista.com/topics/5994/the-coronavirus-disease-covid-19-outbreak/). At the end of 2021 the virus was once again circulating at very high rates, even in countries with relatively high vaccination rates such as the United States and Germany. As rates of new infections increased, some countries in Europe, like Germany and Austria, tightened restrictions once again, specifically targeting those who were not yet vaccinated. However, by spring 2022, rates of new infections had decreased in many countries and restrictions were once again lifted.

This situation is further aggravated by the emergence of novel Covid-19 variants. CDC (covid.cdc.gov/covid-data-tracker/#variant-summary) details its online surveillance of Covid-19 variants. (3) Numerous variants of the virus that causes COVID-19 are being tracked in the United States and globally during this pandemic. As the virus spreads, it has new opportunities to change and may become more difficult to stop. These changes can be monitored by comparing differences in physical traits (such as resistance to treatment) or changes in genetic code (mutations) from one variant to another. By studying each variant and understanding these differences, scientists can monitor, and often predict, whether a variant is more dangerous than others. Scientists can also use this information to track the spread of a variant.

Tierney A. L, Alali W. M, Scott T, Rees-Unwin K. S.’ CITIID-NIHR Bio reference Covid-19 Collaboration, Clark S. J. and Unwin R. D. studied and measured “Levels of Soluble complement regulators predict severity of Covid-19 Symptoms” and published their findings in Front. Immunol. 13:1032331. doi: 10.3389/fimmu.2022.1032331 (4). In the abstract they state:

“The SARS-CoV-2 virus continues to cause significant morbidity and mortality worldwide from COVID-19. One of the major challenges of patient management is the broad range of symptoms observed. While the majority of individuals experience relatively mild disease, a significant minority of patients require hospitalization, with COVID-19 still proving fatal for some. As such, there remains a desperate need to better understand what drives this severe disease, both in terms of the underlying biology, but also to potentially predict at diagnosis which patients are likely to require further interventions, thus enabling better outcomes for both patients and healthcare systems. Several lines of evidence have pointed to dysregulation of the complement cascade as a major factor in severe COVID-19 outcomes. How this is under pinned mechanistically is not known. Here, in the review they have focused on the role of the soluble complement regulators Complement Factor H (FH), its splice variant Factor H-like 1 (FHL-1) and five Factor H-Related proteins (FHR1-5). Using a targeted mass spectrometry approach, we quantified these proteins in a cohort of 188 plasma samples from controls and SARS-CoV-2 patients taken at diagnosis. This analysis revealed significant elevations in all FHR proteins, but not FH, in patients with more severe disease, particularly FHR2 and FHR5 (FHR2: 1.97-fold, p<0.0001; FHR5: 2.4-fold, p<0.0001). Furthermore, for a subset of 77 SARS-CoV-2 +ve patients they also analyzed time course samples taken approximately 28 days post-diagnosis. Here, they see complement regulator levels drop in all individuals with asymptomatic or mild disease, but regulators remain high in those with more severe outcomes, with elevations in FHR2 over baseline levels in this group. These data support the hypothesis that elevation of circulating levels of the FHR family of proteins could predict disease severity in COVID-19 patients, and that the duration of elevation (or lack of immune activation resolution) may be partly responsible for driving poor outcomes in COVID-19.”

The invention details recent advances in Self-Non Self path in host that contributes to immune pathogenesis of Covid-19 and amplifies it to cause global pandemic related morbidity and mortality and method to overcome it.

In the first step, SARS-Cov-2 virus and its variants bind with Factor H and its variants hijack the controlling elements of “C3 amplification loop”. Factor H is recognized as dominant immune regulatory path in that protect host self. This mechanism of Self-protection of host is subverted by Covid-19 and its variants by binding to Factor H to facilitates its entry inside host cells and multiply.

In second step of immune pathogenesis, that occur simultaneously with first step is to hyper activate Factor D or Non self path. By inhibiting the control mechanism of C3 amplification and stimulating or hyper amplifying “C3 amplification loop SARS-Cov-2 act as a “runaway train with failed breaks” contributing to break down products of C3b convertase and its break down products such as C3a and C5a to form anaphylotoxins such as C3a and C5a. They interact with C3ar and C5aR on host immune cells to liberate inflammatory cytokines and contribute to migration of inflammatory cells such as neutrophils to cause multi organ failures.

The above two steps of immune pathogenesis contribute to immune evasion, immune suppression and immune inflammation in the host fluid and tissues. This contributes to global pandemic related morbidity and mortality.

### SUMMARY OF THE INVENTION

The invention details novel prophylaxis, immune therapy and vaccine strategies for SARS-CoV-2 related Covid-19 and its variants. This is based on Recent advances in “Self-Non Self” concepts to boost host immunity.

In first mode of invention, two steps of immune pathogenesis of SARS-CoV-2 and its related Covid-19 and variants are defined that contributes to global pandemic spread and its related morbidity and mortality.

In the first step of immune pathogenesis, immune evasion of SARS-CoV-2 is outlined that allows virus to breakdown the controlling mechanism of “C3 Amplification Loop” and accelerate its amplification.

In the second step of immune pathogenesis of SARS-CoV-2—simultaneously, Factor D is hyper activated that contributes to extreme hyper activation of C3 amplification loop. Immune dysfunction due to extreme hyper activation of C3 amplification loop contributes to breakdown of C3b convertase to C3a and C5a. They react with C3aR and C5aR on immune cells. Together, they form highly inflammatory environment with influx of inflammatory cytokines and cells that facilitate and accelerate the global morbidity and mortality due to Covid-19 and its variants.

In the second mode of the invention we detail a molecular method to target multidimensional immune pathogenesis of SARS-CoV-2 in fluid and tissues with SNS mimicking decoy compound that neutralizes Steps 1-2 disclosed above that will inhibit immune pathogenesis of SARS-CoV-2 and allow adaptive immunity to recover and generate vaccine responses that can be augmented with evolving vaccine strategies in Covid-19 vaccines for variants.

In 2A mode of invention the method is used to boost host immune responses as a prophylaxis in High-risk family members and community members to inhibit SARS-CoV-2 piracy of controlled mechanism of C3 amplification loop by down regulating piracy of fluid and tissue complement regulators as well as the hyper amplification.

In 2B mode of invention the method is targeted to symptomatic patients to inhibit severity of symptoms and spread.

In 2C mode of invention, novel formulation methods are developed with current and evolving Drugs and vaccines by developing strategic combination with SNS mimicking molecule to Overcome Covid-19 pandemics due to its variants.

Most specifically, the invention details sulfonic polymers and its novel formulation methods to inhibit SNS related immune pathogenesis of SARS-CoV-2 and its related morbidity and mortality by targeting Factor H, B and D that contributes to C3 amplification dysfunction in both fluid and host cells to contribute to global pandemic related morbidity and mortality.

AI approach is used in one mode of invention to predict and visualize immune pathogenesis of SARS-CoV-2 contributing to Covid-19 and its pandemic due to its variants.

AI is used in the second mode of invention for the screening and discovery of sulfonic polymers that have SNS Mimicking properties and its novel formulation methods as outlined in the second mode of invention to inhibit SARS-CoV-2 immune piracy and immune dysfunction that contribute to global spread, morbidity and mortality.

AI is used in the third mode of invention to analyze SNS mimicking properties of sulfonic polymers so that it can predict human safety and efficacy before clinical trial.

AI in the fourth mode of invention is used to define multidimensional immune pathogenesis of SARS-CoV-2 related Covid-19 and its variants in targeting with SNS mimicking compound to develop strategic novel formulation methods for clinical trials targeting step 1-2 and its immune pathogenesis of Covid-19 and its variants.

In one embodiment, the invention provides a method for identifying candidate drug compounds for treating Covid-19 variants. In a first step, there is stored a multiplicity of scientific literature documents including immune pathogenesis of SARS-CoV-2 and SARS-CoV-2 patient characteristics and related comorbid conditions on a server. The next step includes generating an analysis module of an artificial intelligence engine which utilizes natural language processing for extracting molecular data from the scientific literature documents and ranking the molecular data based on its relationship with different patient outcomes. Finally, the method includes applying the analysis module to the scientific literature documents for identifying candidate drug compounds from the extracted molecular data with a Self-Non Self (SNS) mimicking decoy function for neutralizing the immune pathogenesis of SARS-CoV-2.

The immune pathogenesis of SARS-CoV-2 includes immune evasion of SARS-CoV-2 virus to breakdown a controlling mechanism of a C3 Amplification Loop and extreme hyper activation of C3 amplification loop wherein the candidate drug compounds inhibit the SARS-CoV-2 controlling mechanism of C3 amplification loop by down regulating piracy of fluid and tissue complement regulators. The immune pathogenesis of SARS-CoV-2 includes SARS-CoV-2 virus hyper activating Factor D which contributes to extreme hyper activation of C3 amplification loop and wherein the candidate drug compound inhibits one or more of Factor H and Factor B and Factor D to disrupt C3 amplification dysfunction. The extreme hyper activation of C3 amplification loop includes a breakdown of C3b convertase to C3a and C5a which react with C3aR and C5aR on immune cells forming a highly inflammatory environment with a corresponding influx of inflammatory cytokines and cells.

The generating step includes extracting molecular data and screening the extracted molecular data for sulfonic polymers, and wherein the identifying step includes rating the screened molecular data for sulfonic polymers based on efficacy in providing a SNS mimicking decoy function. The rating step includes comparing the screened molecular data for sulfonic polymers for efficacy to SARS-CoV-2 patient characteristics to predict human safety as a precursor to conducting clinical trials. The ranked molecular data is correlated with the screened molecular data to select candidate drug compounds for administration in clinical trials.

The generating step includes extracting a sulfonic polymer data set and at least one additional different molecular data sets to provide a multidimensional immune pathogenesis neutralization strategy, wherein a candidate drug compound from the sulfonic polymer data set and an additional candidate drug compound from the at least one additional different molecular data sets are both administered in clinical trials.

The method further includes the step of administering a SARS-CoV-2 vaccine and the screened candidate drug compound for allowing adaptive immunity, for allowing recovery and for generating a positive vaccine responses. The method of claim 6, wherein the method further includes the step of administering the screened candidate drug compound to symptomatic patients for inhibiting the severity of symptoms and limiting patient to patient spread.

The generating step includes extracting molecular data and screening the extracted molecular data for sulfonic polymers, and wherein the identifying step includes rating the screened molecular data for sulfonic polymers based efficacy in providing a SNS mimicking decoy function, and wherein the screened candidate drug compounds are filtered to identify compounds that inhibit Factor H and Factor B and Factor D to disrupt C3 amplification dysfunction.

In a further embodiment of the invention, there is provided a system for identifying candidate drug compounds for treating Covid-19 variants. A server is provided to store a multiplicity of scientific literature documents including immune pathogenesis of SARS-CoV-2 and SARS-CoV-2 patient characteristics and related comorbid conditions. The server further has stored thereon a tangible, non-transitory computer readable medium that is executed by a provided processor. The processor is programmed for generating an analysis module of an artificial intelligence engine which utilizes natural language processing for extracting molecular data from the scientific literature documents and ranking the molecular data based on its relationship with different patient outcomes. The processor is further programmed for applying the analysis module to the scientific literature documents for identifying candidate drug compounds from the extracted molecular data with a Self-Non Self (SNS) mimicking decoy function for neutralizing the immune pathogenesis of SARS-CoV-2.

In another embodiment of the invention, there is provided a device for identifying candidate drug compounds for treating Covid-19 variants. A server is provided to store a multiplicity of scientific literature documents including immune pathogenesis of SARS-CoV-2 and SARS-CoV-2 patient characteristics and related comorbid conditions. An analysis module of an artificial intelligence engine is provided which utilizes natural language processing for extracting molecular data from the scientific literature documents and ranking the molecular data based on its relationship with different patient outcomes. The analysis module is applied to the scientific literature documents for identifying candidate drug compounds from the extracted molecular data with a Self-Non Self (SNS) mimicking decoy function for neutralizing the immune pathogenesis of SARS-CoV-2.

### BRIEF DESCRIPTION OF THE DRAWINGS

The advantages, nature, and various additional features of the invention will appear more fully upon consideration of the illustrative embodiments now to be described in detail in connection with accompanying drawings. In the drawings wherein like reference numerals denote similar components throughout the views:

FIG. 1 is a diagram illustrating an AI based integrated model of immunity in a normal host.

FIG. 2 is a chart amplifying FIG. 1, focusing on the role of the C3 amplification loop and its controlling mechanism to generate normal immunity and what immune dysfunction results when SARS-CoV-2 hijacks the key regulatory molecules. The diagram highlights step 1-2 in C3 amplification loop of Covid-19 and its variants that are related global pandemic spread, morbidity and mortality.

FIG. 3 is a diagram illustrating an AI based SNS mimicking (sulfonic polymer) molecule.

FIG. 4 is a table reporting the results of in-vitro binding activity of SNS mimicking (sulfonic polymers) and complement proteins in serum that are involved in the control of C3 amplification loop and preventing immune evasion and immune inflammation.

FIG. 5 is a diagram illustrating an embodiment of an Artificial Intelligence (AI) tool kit used to develop Self-Non Self modeling for Covid-19 and its emerging variants that accelerate morbidity and mortality in comorbid and immune suppressive conditions such as aging, diabetes, cancer HIV and MTB.

FIG. 6 is a flowchart depicting the various steps that are executed in the method according to one embodiment of the invention.

### DETAILED DESCRIPTION OF THE PREFERRED EMBODIMENTS

The objects of the invention are achieved by applying “AI tool kit of SNS” in Covid-19 and its variants.

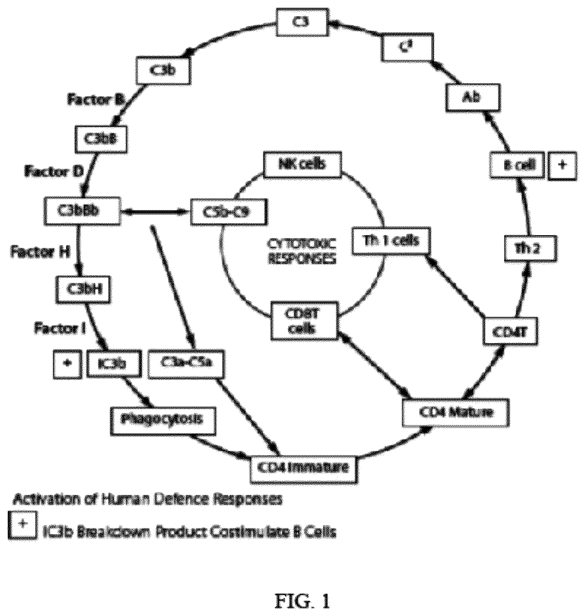

As shown in FIG. 1, AI approach is used to integrate complement based advances (C3: Classical, Lectin and Alternate Complement System) the control earliest immunity development.

C3 are three elements of Complement system where Classical and Lectin based system, join Alternate complement system to take part in C3 amplification process. This process requires application of AI based visual system to visualize flowing of Classical and Lectin system and merging with Alternate complement system. The alternate complement proteins such as Factor H and Factor D are driven by Factor B, Factor D and Factor H. The end objective of such C3 amplification system is to boost immune responses of host by

First by forming C3bBb convertase, this acts as flagging molecule for intruder such as SARS-CoV-2.

Second by cooperation of Factor D with Factor H to form C5b-C9 cytotoxic immune responses so that stimulated immune system only target intruding SARS-CoV-2 and not host.

Third, Factor H binds to highly glycosylated molecules of host cell and provide protection against adverse effects of stimulated immune system.

Fourth, down stream products that are generated due to breakdown of C3bBb, such as C3a-C5a, formation of C5b-C9 and Factor H downstream inactivated iC3b, etc. contribute to stimulate T and B cell adaptive immune responses.

Thus, to generate host immunity, alternate complement system is the most ancient human defense system that has protected mankind from its most ancient time. It is this system that educates, prime early immune responses through cooperation from Classical and Lectin based system and stimulate delayed immune responses with T and B cells when threatened with intruders such as SAS-CoV-2.

The three arms of the complement system, namely, Classical, Lectin and Alternate complement system, should be visualized as a fastmoving train that merge at C3 amplification loop but it is primarily controlled by Alternate complement proteins such as Factor H and Factor D. The balanced modulation and cooperation of Factor D and Factor H boost host immunity while preventing adverse effects on host.

It should be noted that Factor H piracy and Factor D hyper activation contribute to extreme “C3 Hyper amplifications” in immune suppressive comorbid conditions such as HIV, Tuberculosis, cancer, and aging as well as in diabetes that greatly accelerate Morbidity and mortality due to Covid-19 and its variants. all comorbid conditions including aging contribute to extremely hyper amplify C3 amplification loop and their actions are synergistic to cause morbidity, mortality and prevention of host immune responses.

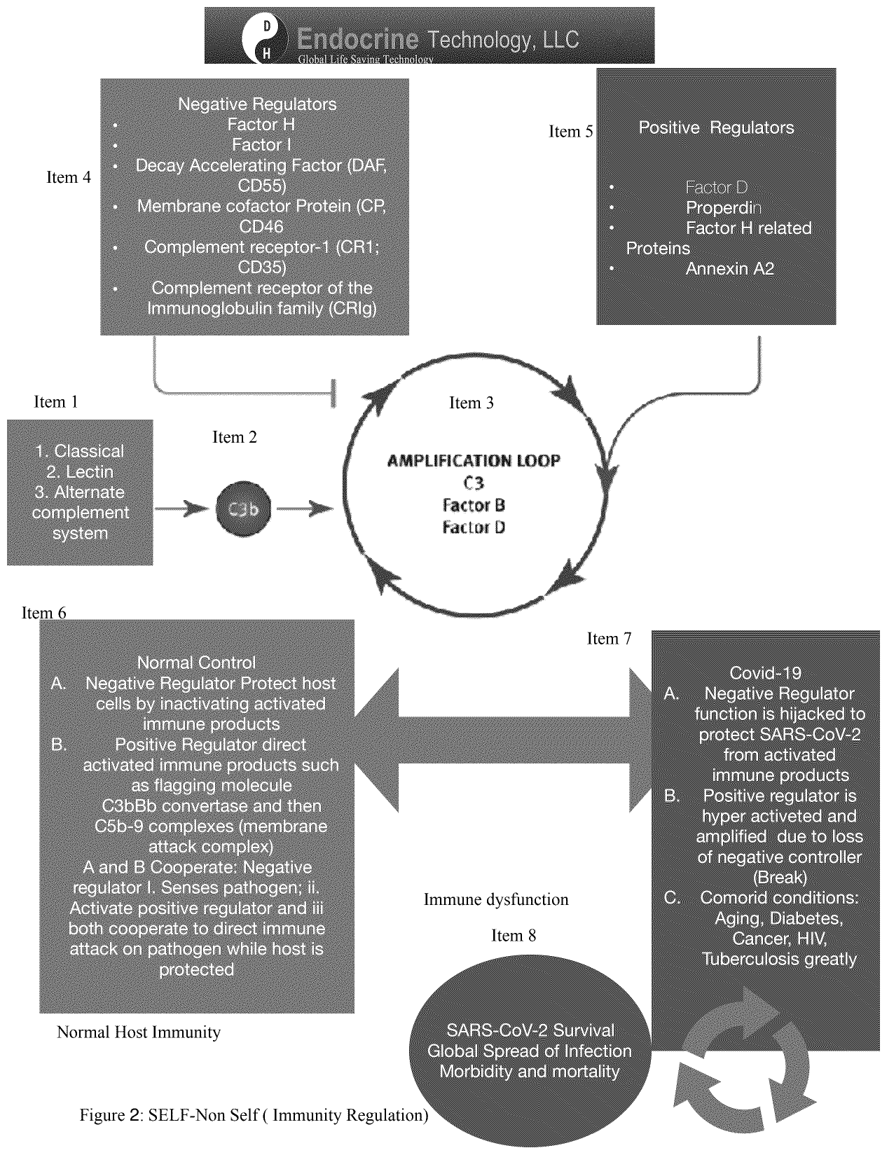

FIG. 2 is a chart providing further amplification of FIG. 1 to focus on the role of C3 amplification. The diagram details the modulation or inhibition of Factor H and Factor D that will inhibit “C3 Amplification loop” to stimulate host immunity by reducing immune suppressive, immune evasive and immune inflammatory behavior of Sars-CoV-2 to allow adequate time to form adaptive immunity to protect host. The inhibition of Factor H and D will also contribute to down regulation of C3 amplification loop that contributes to SARS-CoV-2 related global pandemic related spread, morbidity and mortality.

In the diagram, Item 1 illustrates the three elements of the complement system, namely, the classical, lectin and alternate system converge to form Item 2.

Item 2 is the C3 molecule, which in the presence of Factor B and D form C3 amplification loop as shown in Item 3.

Item 3 is the C3 amplification loop that is regulated by negative and positive regulators.

Item 4 shows the Factor H and I negative regulators and the Complement immune regulators in host cell surfaces. Here, Factor H plays a dominant role in both fluid and on the host cell surface.

Item 5 shows the positive regulators of the C3 amplification loop and includes Factor D, Properdin, Factor H related proteins and Annexing A2. Here, Factor D plays a dominant role.

Item 6 shows normal host immunity generated by a) Sensing of intruding pathogen (Factor H); and b) alerting and activating Factor D of intruding pathogen so that it can be activated. Factor H and D coordinate to form flagging molecule, C3bBb convertase and its subsequent membrane attack complex. This was the host gets protected by Factor H binding to host cell surface where it inactivates activated immune products. Factor D directs immune attack products only to intruding pathogens. Since the most ancient time, this is the way the host is protected from environmental attacks.

Item 7 shows how in Covid-19, SARS-CoV-2 and its variants avoid immune destruction by mining to complement regulators such as Factor H. By inhibiting negative immune regulators, the positive immune regulators are hyper activated. In co-morbid condones, the same underlying pathogenesis leads to extreme hyper activation and host inflammatory syndrome.

Item 8 shows recycling events from Item 7 contribute to immune dysfunction ion leading to survival of SARS-CoV-2, spread, morbidity and mortality at global levels.

As shown in FIG. 2, AI approach is used to track detail sequences of SARS-CoV-2 immune pathogenesis and how it contributes to global pandemic of Covid-19 and its variants.

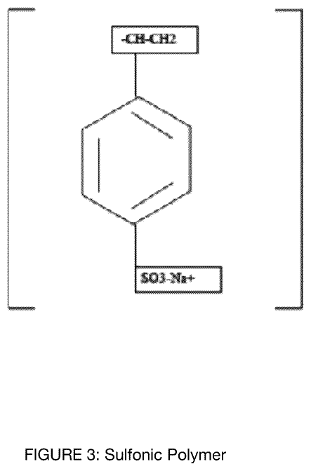

FIG. 3 is a diagram illustrating an AI based SNS mimicking (sulfonic polymer) molecule.

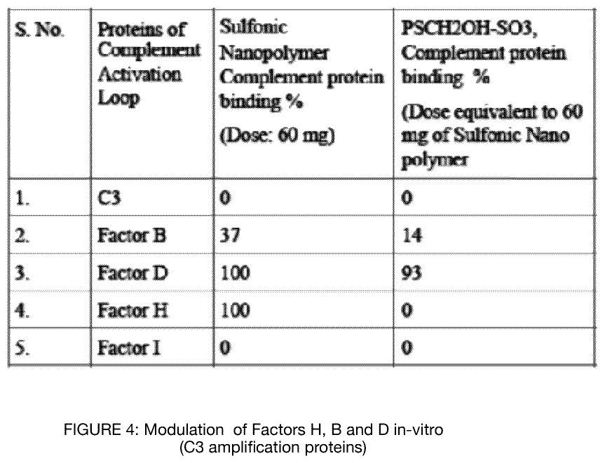

FIG. 4 is a table reporting the results of in-vitro binding activity of SNS mimicking (sulfonic polymers) and complement proteins in serum that are involved in the control of C3 amplification loop and inhibition of immune piracy, immune evasion and immune inflammation.

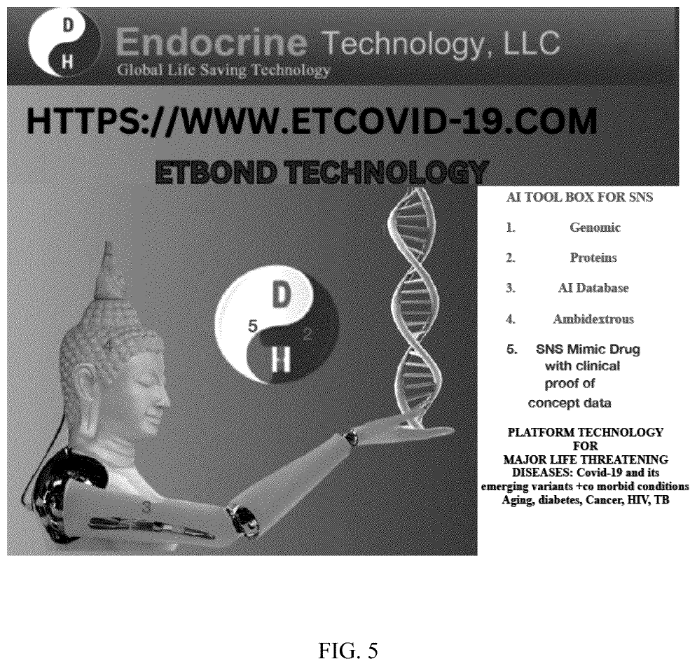

As shown in FIG. 5, The tool kit for AI has several elements listed 1-5 that need to be carefully coordinated and modified with strategic formulation methods to develop appropriate therapy, prophylaxis and vaccine for Covid-19 and its variants.

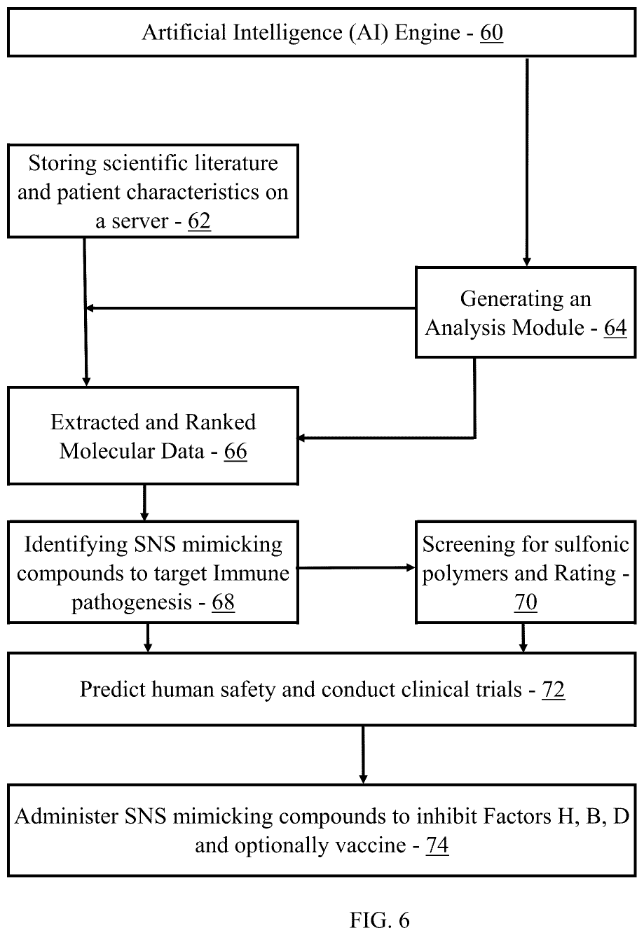

FIG. 6 depicts the processing by an Artificial intelligence engine that analyzes various sources of data to identify SNS mimicking compounds, predict human safety in advance of administering the novel compounds described herein.

More specifically, FIG. 6 shows an Artificial Intelligence Engine in step 60. A variety of data is stored on a server 62. For example, scientific literature documents including immune pathogenesis of SARS-CoV-2 and SARS-CoV-2 patient characteristics and related comorbid conditions. An analysis module is generated in step 64 that utilizes natural language processing for extracting 66 molecular data from the scientific literature documents. The analysis module further is programmed to rank the molecular data based on its relationship with different patient outcomes. The analysis module identifies SNS mimicking compounds to target immune pathogenesis of in step 68. The analysis module is applied to screen for sulfonic polymers and rate for efficacy in step 70. The screened molecular data is compared to the SARS-CoV-2 patient characteristics to predict human safety and conduct clinical trials in step 72. Finally, acceptable SMS mimicking compounds are administered to inhibit Factor H, Factor B, Factor D, or two or more of these, along with optional vaccines in step 74.

At least one server, AI Engine 60 and/or Analysis module 64 may be designed and configured to receive training data. Training data consists of information containing correlations that a machine-learning process may use to model relationships between two or more categories of data elements. Training data may include a plurality of data points, data elements may be correlated by shared existence in a given data entry, by proximity in a given data entry, or data sets combining like data points or data elements. Multiple data entries in training data may be compared or contrasted to reveal trends or correlations between categories of data elements. Such trends or correlations could indicate a possible proportional or other mathematical relationship linking values belonging to the two categories. Multiple categories of data elements may be related in training data according to various correlations; correlations may indicate cause and effect relationships or predictive trends between categories of data elements. These relationships or trends can be used to model structures by machine-learning processes. Training data may be formatted and/or organized by categories of data elements, for instance by associating data elements with one or more descriptors corresponding to categories of data elements. As a non-limiting example, training data may include data entered in standardized forms or scanned or entered by natural language processing to extract data such that entry of a given data element in a given field in a form may be organized. Elements in training data may be linked to descriptors of categories by tags, tokens, or other data elements.

Thus in item 1, focus is on Chromosome 1 at Q32 position. This is the weakest and most vulnerable location in entire genome of over 30,000 genes that contribute to SARS-CoV-2 survival, Covid-19 and its related variants spread and its related morbidity and mortality.

Item 2A, the key protein that is involved in above location that contribute to SARS-CoV-2 survival, spread and its related morbidity and mortality is Factor H. This is a dominant immune regulatory protein that control both fluid as well as complement control proteins located on host cell surface. When typed in as “Factor H and Covid-19” in Pub Med Central National Library of Medical Scientific Database, there are over 1277 articles (ncbi.nlm.nih.gov/pmc/?term=Factor+H+and+Covid-19) (5). A carefully analysis of this data reveal factor H is essential for fluid phase alternative pathway complement control, as well as for protection of host cells.

Item 2B, when typed in as “Factor H and Covid-19 comorbid conditions such as HIV or Cancer or diabetes or tuberculosis, additional articles are revealed that contribute to “C3 Amplification loop abnormalities and cause Covid-19 pandemic spread, morbidity and mortality.

Item 3: Futuristic AI application involves analysis of database of Factor H abnormalities in Covid-19 and its comorbid conditions such as aging, diabetes, cancer and tuberculosis and its relationship with various patient characteristics to develop novel formulation methods for futuristic personalized and precision medicines for Covid-19therapy, prophylaxis and vaccines.

Item 4: Since the utility of Robotic model to deploy personalized consultancy for Covid-19 depends upon the deep knowledge of medicine, recent advances in SNS modeling and industry efforts to develop novel drugs, machine learning is required to train AI model for predictive and personalized Covid-9 therapy and vaccines.

Item 5: SNS mimicking compound should ideally down regulate both Factor H and Factor D in a balanced way to avoid adverse effects of Factor H on host and should lead to successfully cytotoxic immune therapy while boosting host immunity. Most importantly, by inhibiting interactions of factor H with SARS-CoV-2 a therapeutic modulation of SARS-CoV-2 is feasible to prevent its subversive actions are feasible.

For fastest deployment of therapeutic advances in Covid-19 one need to consider individual patient's need in life science to expediently advance and tailor Covid-19 immune therapy, prophylaxis and vaccination at global levels. FDA has several paths that can be used to develop compassion based approaches since SNS mimicking compound has extensive human safety data.

From a Biotechnology perspective, AI modeling can be applied to predict human safety and appropriate formulation method to advance to phase 1-3 clinical trial to further determine safety and efficacy of futuristic therapies.

Structural Formula of SNS mimicking Sulfonic Polymer that come with varying molecular size from 100 nm to 25 micron and number of polymers from N 1-100.

Description: SNS Mimicking compound is sodium polystyrene sulfonate with variable N reflecting N-1 as monomer and N-100 as polymer with varying properties based on molecular weight.

It is benzene, diethenyl-polymer, with ethenylbenzene, sulfonated, sodium salt and has the structural formula as in Figure. polystyrene sulfonate (SPS), a cation-exchange resin prepared in the sodium phase with an in-vitro exchange capacity of approximately 3.1 mEq (in vivo approximately 1 mEq) of potassium per gram of the drug. The sodium content is approximately 100 mg (4.1 mEq) per gram of the drug. Polystyrene sulfonate (PSS) is a negatively charged polyelectrolyte bearing sulfonate groups. Due to the very low pKa of the sulfonate group (−2.1) PSS is fully charged in a wide range of pH and ionic strength conditions that contributes to its SNS binding with Factor H and Factor D.

Proposed Dose and the route of administration for Immune prophylaxis of healthy subjects: Suspension of the drug should be freshly prepared and not stored beyond 24 hours. The average daily dose for Covid-19 prophylaxis or for host immunity boosting effects is 250 mg twice a day. Each dose is administered as warm fluid such as 0.85% saline warmed to body temperature in 50-100 ml fluid. The fluid should be agitated gently to prevent precipitation. Better formulation method for inhalation will help counter Pulmonary infection.

Proposed dose for Immune prophylaxis for SRS-CoV-2 exposed patients, with comorbid conditions such as aging, diabetes, cancer and tuberculosis is 3-5 mg/kg body weight given intramuscularly either in suitable sterile formulation or by intravenous injections. The later approach is particularly well suited in hospitalized patients that are extremely ill and constantly monitored for vital signs and fluid replacements. Actual clinical trial may need to be conducted with existing and evolving drugs to determine the exact formulation method, dose and its frequency.

Proposed dose with Covid-19 vaccines and its variants: Vaccine is combined with 50-100 mcg of sulfonic polymer to study vaccine responses with or without sulfonic polymers.

### Contraindications

It is contraindicated in patients with hypokalemia or those patients who are hypersensitive to it or in extremely rare patients with Factor H Deficiency.

Drug Interactions Antacids: The simultaneous oral administration of drug with non-absorbable cation-donating antacids and laxatives such as magnesium hydroxide and aluminum carbonate. This reaction generally occurs in significant way if given in extreme high dosage. In the recommended dose no interactions noted.

Digitalis: If the medicine is given in extreme large dosage, the toxic effects of digitalis on the heart, especially various ventricular arrhythmias and A-V nodal dissociation, are likely to be exaggerated by hypokalemia, even in the face of serum digoxin concentrations in the “normal”.

The binding characteristics of C3, Factor B, Factor D, Factor H and Factor I proteins were studied for sulfonic nano polymer and other polymers. NHS with buffer was used as normal control.

The dose of polymers used was standardized.

The NHS in (1:4) dilution with each of above polymers was incubated at 37 degrees C in the water bath with gentle agitation for half hours to 1 hour durations in a series of experiments. The binding activity of complement proteins in the serum was analyzed in duplicate and mean result is depicted. Below are the key results.

### SNS Mimicking Compound: Sulfonic Polymers

In-Vitro Data for Factor D: A. The drug application is based on the fact that as shown above it is a complement inhibitor of Factor D that will inhibit relentlessly activated C3 amplification loop whose downstream products are responsible for C3a-C5a mediated inflammatory responses.

In-vitro Data for Factor H: The drug also inhibits Factor H. As shown above, Factor H is a dominant immune regulatory molecule of Alternate complement system that protect host in both at fluid and over cell membrane. Factor H is 150 kD key immune regulatory protein of Alternate Complement System that is abundantly expressed in GBM Factor H and its downstream regulatory proteins, cause hyper activation uncontrolled activation of Factor D that is directed to host proteins in TME. This sets in repeated auto amplification of complement system contributing complement hyper inflammation and TME inflammation that progress to drug resistance and metastasis.

In vivo data in mice, it inhibits Complement system and CH 50 activity at 0.5 mg/kg body weight In-vivo Mice efficacy data in Inflammatory Disorders: Our initial model studied was Type 1 Non Obese Diabetic (NOD) mice as an autoimmune or inflammatory model of Type 1 Diabetes. This has been used successfully to inhibit inflammation in small animal models including Non Obese Diabetes Mice for the cure of diabetes. As compared to control 5-9 days, the inflammatory mediators were suppressed allowing prolongation of islet cell Graft for up to 80 days.

Currently there is no comparator therapy to modulate Factor H and Factor D function that has extensive human safety data. A number of drug companies are trying to develop such therapies as a way to target C3 amplification loop and are at preclinical levels.

Supplement text details experiments inhibiting Factor H and Factor D are attached.

### Drug Development Plan

Formulation strategy to reduce adverse effects and improve therapeutic efficacy is the part of drug development effort requiring immense input from scientists and international sources. Complexity of medical sciences, resistance to new ideas amongst experts, financial motives, legal complexity, lack of resources, and making life saving drug available at accessible prices are the critical issues in development of new therapeutic applications. Introduction of an established drug that is global, has proven safety, is in keeping with recent advances in “Genomics and fundamentals of immunology” and pertains to life saving application with extensive third party preclinical support allows us to have an AI strategy to over comes many of the hurdles that are inherent and draw attention of global experts as to what can be done to reduce complexity of medical care in Covid-19 therapy and vaccine studies.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, and systems according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention as set forth in the appended claims.

### REFERENCES

Throughout this application, various references describe the state of the art to which this invention pertains. The contents of these documents are hereby incorporated into the present application by reference thereto.
1. Xiao M. T., et al, Emerging role of complement in Covid-19 and other respiratory diseases, Cellular and Molecular Life sciences, 81:94, 2024.
2. statista.com/statistics/1093256/novel-coronavirus-2019ncov-deaths-worldwide-by-country/
3. covid.cdc.gov/covid-data-tracker/#variant-summary
4. Tierney A. L, et al, Levels of Soluble complement regulators predict severity of Covid-19 Symptoms, Front. Immunol. 13:1032331 (2022).
5. ncbi.nlm.nih.gov/pmc/?term=Factor+H+and+Covid-19.

What is claimed is:

1. A method for identifying candidate drug compounds for treating Covid-19 variants comprising the steps of:
storing a multiplicity of scientific literature documents including immune pathogenesis of SARS-CoV-2 and SARS-CoV-2 patient characteristics and related comorbid conditions on a server;
generating an analysis module of an artificial intelligence engine which utilizes natural language processing for extracting molecular data from the scientific literature documents and ranking the molecular data based on its relationship with different patient outcomes; and
applying the analysis module to the scientific literature documents for identifying candidate drug compounds from the extracted molecular data with a Self-Non Self (SNS) mimicking decoy function for neutralizing the immune pathogenesis of SARS-CoV-2.

2. The method according to claim 1, wherein the immune pathogenesis of SARS-CoV-2 includes immune evasion of SARS-CoV-2 virus to breakdown a controlling mechanism of a C3 Amplification Loop and wherein the candidate drug compounds inhibit the SARS-CoV-2 controlling mechanism of C3 amplification loop by down regulating piracy of fluid and tissue complement regulators.

3. The method according to claim 2, wherein the immune pathogenesis of SARS-CoV-2 includes SARS-CoV-2 virus hyper activating Factor D which contributes to extreme hyper activation of C3 amplification loop and wherein the candidate drug compound inhibits one or more of Factor H and Factor B and Factor D to disrupt C3 amplification dysfunction.

4. The method according to claim 3, wherein extreme hyper activation of C3 amplification loop includes a breakdown of C3b convertase to C3a and C5a which react with C3aR and C5aR on immune cells forming a highly inflammatory environment with a corresponding influx of inflammatory cytokines and cells.

5. The method of claim 4, wherein the generating step includes extracting molecular data and screening the extracted molecular data for sulfonic polymers, and wherein the identifying step includes rating the screened molecular data for sulfonic polymers based on efficacy in providing a SNS mimicking decoy function.

6. The method of claim 5, wherein the rating step includes comparing the screened molecular data for sulfonic polymers for efficacy to SARS-CoV-2 patient characteristics to predict human safety as a precursor to conducting clinical trials.

7. The method of claim 6, wherein the ranked molecular data is correlated with the screened molecular data to select candidate drug compounds for administration in clinical trials.

8. The method of claim 6, wherein the generating step includes extracting a sulfonic polymer data set and at least one additional different molecular data sets to provide a multidimensional immune pathogenesis neutralization strategy, wherein a candidate drug compound from the sulfonic polymer data set and an additional candidate drug compound from the at least one additional different molecular data sets are both administered in clinical trials.

9. The method of claim 6, wherein the method further includes the step of administering a SARS-CoV-2 vaccine and the screened candidate drug compound for allowing adaptive immunity, for allowing recovery and for generating a positive vaccine response.

10. The method of claim 6, wherein the method further includes the step of administering the screened candidate drug compound to symptomatic patients for inhibiting the severity of symptoms and limiting patient to patient spread.

11. The method of claim 2, wherein the generating step includes extracting molecular data and screening the extracted molecular data for sulfonic polymers, and wherein the identifying step includes rating the screened molecular data for sulfonic polymers based efficacy in providing a SNS mimicking decoy function, and wherein the screened candidate drug compounds are filtered to identify compounds that inhibit Factor H and Factor B and Factor D to disrupt C3 amplification dysfunction.
